(19) United States
(12) Patent Application Publication
(10) Pub. No.: US 2010/0189763 A1
(43) Pub. Date: Jul. 29, 2010

(54) CONTROLLED RELEASE SYSTEMS FROM POLYMER BLENDS

(76) Inventor: Heather Nettles, Bessemer, AL (US)

Correspondence Address: Ballard Spahr LLP, SUITE 1000, 999 PEACHTREE STREET, ATLANTA, GA, 30309-3915, US

(21) Appl. No.: 12/692,027

(22) Filed: Jan. 22, 2010

Related U.S. Application Data
(60) Provisional application No. 61/146,980, filed on Jan. 23, 2009.

(51) Int. Cl. A61K 47/34; A61K 9/14; A61F 2/00

(52) U.S. Cl. 424/426; 514/772.3; 514/772.7; 424/486

(57) ABSTRACT

Described herein are improved microparticles. In one aspect, the microparticles comprise a first polymer and a second polymer wherein the second polymer is different than the first polymer. In further aspects, the microparticles comprise a bioactive agent encapsulated therein.

### CROSS-REFERENCE TO RELATED APPLICATIONS

This application is based upon and claims the benefit of priority from prior U.S. Provisional Application No. 61/146,980, filed Jan. 23, 2009, which is incorporated herein by reference.

### BACKGROUND

In order for a bioactive agent to work effectively, it must be delivered to a subject in a way that is both safe and effective. An ideal pharmacokinetic profile of a bioactive agent is one which allows for therapeutic concentrations of the bioactive agent to be reached in a subject, while not exceeding the maximum tolerable dose. For certain pharmacological applications, concentrations of the bioactive agent should remain at a therapeutic level for an extended period of time until the desired therapeutic result is achieved.

Unfortunately, conventional routes for administering bioactive agents often do not provide ideal pharmacokinetic profiles, especially for bioactive agents that display high toxicity and/or narrow therapeutic windows. It is known in the art that one way of affecting a pharmocokinetic profile of a bioactive agent is to encapsulate the bioactive agent in a controlled release system. The controlled release system can degrade over time, thereby releasing the bioactive agent according to a release profile that is influenced by the controlled release system.

The release profile or release rate for a bioactive agent may be desired to be different depending on the targeted therapeutic result. Oftentimes, a controlled release system may not provide for a desired release profile, and in some instances can even result in an undesirable release profile. As such, a need exists for controlled release systems and methods for the manufacture thereof that can substantially affect properties of the controlled release system, which can depend on the composition of the controlled release system itself. These needs and other needs are satisfied by the present invention.

### SUMMARY

Described herein are controlled release systems comprising a mixture of polymers, wherein at least two of the polymers in the mixture are different. In one aspect, the properties of the controlled release system can be modulated by selecting the polymer, or a desired property thereof, in the mixture of polymers, to provide a desired property for the controlled release system (e.g., a degradation profile).

In one aspect, the controlled release system comprises a polymer matrix comprising a first polymer and a second polymer that is different from the first polymer; and bioactive agent encapsulated in the polymer matrix

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

FIG. 1 is a plot of in vitro release curves of mixed-polymer formulations from Example 1.

FIG. 2 is a plot of in vitro release curves of mixed-polymer formulations from Example 2.

### DETAILED DESCRIPTION

Before the present compounds, compositions, composites, articles, devices, methods, or uses are disclosed and described, it is to be understood that the aspects described below are not limited to specific compounds, compositions, composites, articles, devices, methods, or uses as such may, of course, vary. It is also to be understood that the terminology used herein is for the purpose of describing particular aspects only and is not intended to be limiting. In this specification and in the claims that follow, reference will be made to a number of terms that shall be defined to have the following meanings:

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

It must be noted that, as used in the specification and the appended claims, the singular forms “a,” “an” and “the” include plural referents unless the context clearly dictates otherwise. Thus, for example, reference to “a bioactive agent” includes mixtures of two or more such agents, and the like.

“Optional” or “optionally” means that the subsequently described event or circumstance can or cannot occur, and that the description includes instances where the event or circumstance occurs and instances where it does not.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

A weight percent of a component, unless specifically stated to the contrary, is based on the total weight of the formulation or composition in which the component is included.

The term “microparticle” is used herein to refer generally to a variety of structures having sizes from about 10 nm to 2000 microns (2 millimeters) and includes microcapsule, microsphere, nanoparticle, nanocapsule, nanosphere as well as particles, in general, that are less than about 2000 microns (2 millimeters). In one aspect, a bioactive agent is encapsulated in the microparticle.

The term “biocompatible” refers a substance that is substantially non-toxic to a subject.

“Biodegradable” is generally referred to herein as a material that will erode to soluble species or that will degrade under physiologic conditions to smaller units or chemical species that are, themselves, non-toxic (biocompatible) to the subject and capable of being metabolized, eliminated, or excreted by the subject.

A “bioactive agent” refers to an agent that has biological activity. The biological agent can be used to treat, diagnose, cure, mitigate, prevent (i.e., prophylactically), ameliorate, modulate, or have an otherwise favorable effect on a disease, disorder, infection, and the like. A “releasable bioactive agent” is one that can be released from a disclosed controlled release system. Bioactive agents also include those substances which affect the structure or function of a subject, or a pro-drug, which becomes bioactive or more bioactive after it has been placed in a predetermined physiological environment.

Disclosed are compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a number of different polymers and agents are disclosed and discussed, each and every combination and permutation of the polymer and agent are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed. The herein disclosed intrinsic viscosity measurements were performed at 30° C. from polymer solutions prepared at a concentration of 0.5 g/dL in chloroform.

In one aspect, the present disclosure relates to micropartices and methods of making the controlled release systems which allow for a desired release profile for the controlled release system to be achieved. Oftentimes when a controlled release system comprises a single polymer, the controlled release system may not demonstrate the desired release profile. The present disclosure relates to tailoring the release profile of a controlled release system by using a mixture of particles to produce the controlled release system.

In general, the controlled release systems can by any controlled release system. In one aspect, the controlled release system comprises a bioactive agent that can be released from the system. Non-limiting examples of controlled release systems include, for example, microparticles, bioactive agent-loaded rods, implant devices, among other devices.

Generally, the disclosed controlled release systems comprise a polymer matrix comprising a first polymer and a second polymer that is different from the first polymer; and bioactive agent encapsulated in the polymer matrix. The term “polymer matrix” as used herein is intended to refer a portion (or all) of the controlled release system which comprises the polymer mixture. The polymer matrix does not necessarily, but can, comprise cross-linked or intertwined polymer chains. In one aspect, the polymer matrix is a polymer composition, wherein the polymer composition encapsulates the bioactive agent. In a further aspect, portions of the polymer matrix can comprise only one of the first and second polymer. Thus, the controlled release system polymer matrix need not be homogenous, although in another aspect the polymer matrix can be homogenous.

The first and second polymer can be present in the controlled release system in any desired ratio, which is the weight ratio of the first polymer to the second polymer. In one aspect, the ratio of the first polymer to the second polymer is from about 90:10 to about 40:60, including ratios without limitation of about 85:15, 80:20, 70:30, 75:25, 65:35, and 50:50, among others. In addition, more than two polymers can be present in a blend, for example, 3, 4, 5, or more polymers can be present.

In one aspect, the first and second polymers have at least one different property. Depending on the desired degradation profile of the controlled release system, a wide variety of properties can be different among the polymers, including without limitation, chemical composition, viscosity (e.g., intrinsic viscosity), molecular weight, thermal properties, such as glass transition temperature (T_{g}), the chemical composition of a non-repeating unit therein, such as an end group, degradation rate, hydrophilicity, porosity, density, or a combination thereof. In one aspect, the first polymer and the second polymer have different degradation rates in an aqueous medium. In one aspect, a degradation profile of a controlled release system is selected, and a combination of polymers having properties that, when combined, are believed to achieve the selected degradation profile are used to make the controlled release system.

In one aspect, the first polymer and the second polymer have one or more different non-repeating units, such as, for example, an end group, or a non-repeating unit in the backbone of the polymer. In a further aspect, the first polymer and the second polymer have one or more different end groups. For example, the first polymer can have a more polar end group than one or more end group(s) of the second polymer. Thus, in this aspect, the first polymer will typically be more hydrophilic and thus lead to faster water uptake, relative to a controlled release system comprising the second polymer (with the less polar end group) alone. In a specific aspect, the first polymer can have one or more carboxylic acid end groups, and the second polymer can have one or more ester end groups.

In another aspect, the first polymer and the second polymer have different molecular weights. In one aspect, the first polymer has a molecular weight that is at least about 3000 Daltons greater than the molecular weight of the second polymer. The molecular weight can have any suitable value, which can, in various aspects, depend on the desired properties of the controlled release system. If, for example, a controlled release system having high mechanical strength is desired, at least one of the polymers can have a high molecular weight. In this example, if it is also desired that the controlled release system have short term release capability (e.g., less than about 2 weeks), then a lower molecular weight polymer can be combined with the high molecular weight polymer. In this aspect, the high molecular weight polymer will typically provide good structural integrity for the controlled release system, while the lower molecular weight polymer can provide short term release capability.

In a similar aspect, one of the polymers can exhibit a glass-transition temperature that is less than the glass-transition temperature exhibited by the other polymer. Thus, for example, a polymer having good thermal stability can be combined with another polymer which might not have good thermal stability but has another desirable property, thereby enabling the composite controlled release system to exhibit properties of both polymers. In a specific example, one of the polymers can exhibit a glass-transition temperature that is from about 5° C. to about 50° C. less than the glass-transition temperature exhibited by the other polymer.

Any combination of the above properties can be used, with any appropriate combination of polymers. It is also understood that the controlled release system can comprise just two, or more than two polymers, including for example controlled release systems having three or more polymers in the polymer matrix.

In general, a wide variety of polymers can be used to achieve the intended results herein. The polymers used can be biocompatible and/or biodegradable. In one aspect, as discussed above, the desired release profile of the bioactive agent can influence the selection of the polymer, or a desired property thereof. A biocompatible polymer, for example, can be selected so as to release or allow the release of a bioactive agent therefrom at a desired lapsed time after the controlled release system has been administered to a subject. For example, the polymer can be selected to release or allow the release of the bioactive agent prior to the bioactive agent beginning to diminish its activity, as the bioactive agent begins to diminish in activity, when the bioactive agent is partially diminished in activity, for example at least 25%, at least 50% or at least 75% diminished, when the bioactive agent is substantially diminished in activity, or when the bioactive agent is completely gone or no longer has activity.

When the first and/or second polymer is a biodegradable polymer, the controlled release system can be formulated so as to degrade within a desired time interval, once present in a subject. In some aspects, the time interval can be from about less than one day to about 1 month. Longer time intervals can extend to 6 months, including for example, polymer matrices that degrade from about ≧0 to about 6 months, or from about 1 to about 6 months. In other aspects, the polymer can degrade in longer time intervals, up to 2 years or longer, including, for example, from about ≧0 to about 2 years, or from about 1 month to about 2 years.

Non-limiting examples of the first and/or second polymer include polyesters, polyhydroxyalkanoates, polyhydroxybutyrates, polydioxanones, polyhydroxyvalerates, polyanhydrides, polyorthoesters, polyphosphazenes, polyphosphates, polyphosphoesters, polydioxanones, polyphosphoesters, polyphosphates, polyphosphonates, polyphosphates, polyhydroxyalkanoates, polycarbonates, polyalkylcarbonates, polyorthocarbonates, polyesteramides, polyamides, polyamines, polypeptides, polyurethanes, polyalkylene alkylates, polyalkylene oxalates, polyalkylene succinates, polyhydroxy fatty acids, polyacetals, polycyanoacrylates, polyketals, polyetheresters, polyethers, polyalkylene glycols, polyalkylene oxides, polyethylene glycols, polyethylene oxides, polypeptides, polysaccharides, or polyvinyl pyrrolidones. Other non-biodegradable but durable polymers include without limitation ethylene-vinyl acetate co-polymer, polytetrafluoroethylene, polypropylene, polyethylene, and the like. Likewise, other suitable non-biodegradable polymers include without limitation silicones and polyurethanes.

In a further aspect, the polymer can be a poly(lactide), a poly(glycolide), a poly(lactide-co-glycolide), a poly(caprolactone), a poly(orthoester), a poly(phosphazene), a poly(hydroxybutyrate) or a copolymer containing a poly(hydroxybutarate), a poly(lactide-co-caprolactone), a polycarbonate, a polyesteramide, a polyanhydride, a poly(dioxanone), a poly(alkylene alkylate), a copolymer of polyethylene glycol and a polyorthoester, a biodegradable polyurethane, a poly(amino acid), a polyamide, a polyesteramide, a polyetherester, a polyacetal, a polycyanoacrylate, a poly(oxyethylene)/poly(oxypropylene) copolymer, polyacetals, polyketals, polyphosphoesters, polyhydroxyvalerates or a copolymer containing a polyhydroxyvalerate, polyalkylene oxalates, polyalkylene succinates, poly(maleic acid), and copolymers, terpolymers, combinations, or blends thereof.

In a still further aspect, useful biocompatible polymers are those that comprise one or more residues of lactic acid, glycolic acid, lactide, glycolide, caprolactone, hydroxybutyrate, hydroxyvalerates, dioxanones, polyethylene glycol (PEG), polyethylene oxide, or a combination thereof. In a still further aspect, useful biocompatible polymers are those that comprise one or more residues of lactide, glycolide, caprolactone, or a combination thereof.

In one aspect, useful biodegradable polymers are those that comprise one or more blocks of hydrophilic or water soluble polymers, including, but not limited to, polyethylene glycol, (PEG), or polyvinyl pyrrolidone (PVP), in combination with one or more blocks another biocompabible or biodegradable polymer that comprises lactide, glycolide, caprolactone, or a combination thereof.

In specific aspects, the biodegradable polymer can comprise one or more lactide residues. To that end, the polymer can comprise any lactide residue, including all racemic and stereospecific forms of lactide, including, but not limited to, L-lactide, D-lactide, and D,L-lactide, or a mixture thereof. Useful polymers comprising lactide include, but are not limited to poly(L-lactide), poly(D-lactide), and poly(DL-lactide); and poly(lactide-co-glycolide), including poly(L-lactide-co-glycolide), poly(D-lactide-co-glycolide), and poly(DL-lactide-co-glycolide); or copolymers, terpolymers, combinations, or blends thereof. Lactide/glycolide polymers can be conveniently made by melt polymerization through ring opening of lactide and glycolide monomers. Additionally, racemic DL-lactide, L-lactide, and D-lactide polymers are commercially available. The L-polymers are more crystalline and resorb slower than DL-polymers. In addition to copolymers comprising glycolide and DL-lactide or L-lactide, copolymers of L-lactide and DL-lactide are commercially available. Homopolymers of lactide or glycolide are also commercially available.

When the biodegradable polymer is poly(lactide-co-glycolide), poly(lactide), or poly(glycolide), the amount of lactide and glycolide in the polymer can vary. In a further aspect, the biodegradable polymer contains 0 to 100 mole %, 40 to 100 mole %, 50 to 100 mole %, 60 to 100 mole %, 70 to 100 mole %, or 80 to 100 mole % lactide and from 0 to 100 mole %, 0 to 60 mole %, 10 to 40 mole %, 20 to 40 mole %, or 30 to 40 mole % glycolide, wherein the amount of lactide and glycolide is 100 mole %. In a further aspect, the biodegradable polymer can be poly(lactide), 95:5 poly(lactide-co-glycolide) 85:15 poly(lactide-co-glycolide), 75:25 poly(lactide-co-glycolide), 65:35 poly(lactide-co-glycolide), or 50:50 poly(lactide-co-glycolide), where the ratios are mole ratios. In a specific aspect, the first and second polymers are both poly(lactide-co-glycolide) polymers. In a further specific aspect, the ratio of lactide to glycolide is from about 90:10 to about 40:60. In still a further specific aspect, the ratio of lactide to glycolide is from about 85:15 to about 50:50.

In a further aspect, the polymer can be a poly(caprolactone) or a poly(lactide-co-caprolactone). In one aspect, the polymer can be a poly(lactide-caprolactone), which, in various aspects, can be 95:5 poly(lactide-co-caprolactone), 85:15 poly(lactide-co-caprolactone), 75:25 poly(lactide-co-caprolactone), 65:35 poly(lactide-co-caprolactone), or 50:50 poly(lactide-co-caprolactone), where the ratios are mole ratios.

It is understood that any combination of the aforementioned biodegradable polymers can be used, including, but not limited to, copolymers thereof, mixtures thereof, or blends thereof. Likewise, it is understood that when a residue of a biodegradable polymer is disclosed, any suitable polymer, copolymer, mixture, or blend, that comprises the disclosed residue, is also considered disclosed. To that end, when multiple residues are individually disclosed (i.e., not in combination with another), it is understood that any combination of the individual residues can be used.

Non-limiting specific examples of polymer mixtures for use in a disclosed controlled release system, with their targeted delivery profile, include those mixtures listed in Table 1.

TABLE 1
Exemplary Polymer Mixtures for controlled release systems.

| | Second | First polymer: | Targeted delivery |
|---|---|---|---|
| First polymer | polymer | Second Polymer | profile |
| 8515 DLG 4.5E | 8515 DLG 6A | 50:50 | 4-6 months |
| | | | delivery |
| 7525 DLG 7A | 6535 DLG 2E | 85:15 | 4-6 months |
| | | | delivery |
| 7525 DLG 5E | 6535 DLG 4A | 80:20 | 4-6 months |
| | | | delivery |
| 8515 DLG 5A | 7525 DLG 5E | 50:50 | 4-6 months |
| | | | delivery |
| 8515 DLG 7A | 7525 DLG 7E | 50:50 | 4-6 months |
| | | | delivery |
| 6535 DLG 4A | 2000 MW | various ratios | about 1 month |
| | DLPL | | delivery |
| 5050 DLG 4A | 2000 MW | various ratios | about 1 month |
| | DLPL | | delivery |
| 6535 DLG 4A | 5050 DLG 2A | various ratios | about 1 month |
| | | | delivery |
| 5050 DLG 4A | 5050 DLG 2A | various ratios | about 1 month |
| | | | delivery |

The following example defines the nomenclature used for the polymers in Table 1. The polymer, (8515 DLG 4.5E) refers to poly(D-lactide-co-glycolide), wherein the lactide to glycolide mole ratio is 85:15, wherein the copolymer exhibits an intrinsic viscosity of 0.45 dL/g, and wherein the copolymer comprises an ester (E) end group. The abbreviated (A) refers to an acid (e.g. a carboxylic acid) end group. The polymer 2000 MW DLPL refers to poly(D,L-lactide) having a molecular weight of about 2000 Daltons. The molecular weight of the polymers can be a measured value, or a value provided by a commercial supplier. As such, it is understood that molecular weights may only be close to the molecular weight of the polymer.

The first and second polymers can have a wide range of molecular weights. In one aspect, the molecular weights can range from about 1,000 to about 50,000 g/mol, from about 1,000 to about 20,000 g/mol, from about 1,000 to about 10,000 g/mol, or from about 1,000 to about 5,000 g/mol. In a further aspect, the first and second polymer can differ by molecular weight and/or by any other property disclosed herein.

In a specific aspect, the first polymer is poly(lactide), and the second polymer is poly(lactide-co-glycolide) having a ratio of lactide to glycolide of from about 90:10 to about 50:50, for example 75:25; wherein the ratio of the first polymer to the second polymer is from about 90:10 to about 50:50, for example, 75:25. In a further specific aspect, the first polymer is poly(lactide), the second polymer is poly(lactide-co-glycolide) having a ratio of lactide to glycolide of from about 75:25 to about 50:50; wherein the ratio of the first polymer to the second polymer is from about 90:10 to about 50:50, for example, 75:25. In a further specific aspect, the first polymer is poly(lactide), and the second polymer is polyethylene glycol (PEG) having a molecular weight of about 1500 Daltons; wherein the ratio of the first polymer to the second polymer is from about 90:10 to about 50:50, for example, 75:25.

In one aspect, the controlled release system is a microparticle. The microparticle can be any microparticle produced from the disclosed polymer mixtures. The micropaticles can have a wide variety of shapes and sizes. In one aspect, the disclosed microparticles can have an average or mean particle size of from about 20 microns to about 125 microns. In one embodiment the range of mean particle size is from about 40 microns to about 90 microns. In another embodiment the range of mean particle sizes is from about 50 microns to about 80 microns. Particle size distributions are measured by laser diffraction techniques known to those of skill in the art.

The microparticle can modulate the release of the bioactive agent, depending on the amount of bioactive agent present in the first aqueous phase. For example, the microparticle can comprise 1%, 5%, 10%, 20%, 30%, 40%, 50%, 60%, 70%, 80%, 90%, 95% by weight bioactive agent, relative to the weight of the microparticle, including any range between the disclosed percentages.

The microparticles can be made from the polymer mixtures using methods known in the art, including, for example, those methods disclosed in U.S. Patent Publication No. 2007/0190154 to Zeigerson, published Aug. 16, 2007, and U.S. Pat. No. 5,407,609 to Tice et al., both of which are incorporated herein in their entirety by this reference for teachings of microparticle preparation methods. As will be apparent, depending upon processing conditions, the polymer used as a starting material in the admixing step may or may not be the same polymer present in the final microparticle. For example, the polymer during processing may undergo polymerization or depolymerization reactions, which ultimately can produce a different polymer that was used prior to processing. Thus, the term “polymer” as used herein covers the polymers used as starting materials as well as the final polymer present in the device produced by the methods described herein. Methods for making microparticles can be used in combination with the drying methods and dyring parameters described above.

It will be apparent that, in one aspect, an advantage of using the disclosed polymer mixtures in controlled release system production is that a desired product performance, such as a degradation profile, can be substantially achieved in a single controlled release system production process, rather than preparing multiple controlled release system products and combining the controlled release systems in another mixing step.

A wide variety of bioactive agents can be used with the methods described herein. In one aspect, the bioactive agent can be a releasable bioactive agent, i.e., a bioactive agent that can be released from the controlled release system into adjacent tissues or fluids of a subject. In certain aspects, the bioactive agent can be in or on the controlled release system.

Various forms of the bioactive agent can be used, which are capable of being released from the controlled release system into adjacent tissues or fluids. To that end, a liquid or solid bioactive agent can be incorporated into the controlled release system described herein. The bioactive agents are at least very slightly water soluble, and preferably moderately water soluble. The bioactive agents can include salts of the active ingredient. As such, the bioactive agents can be acidic, basic, or amphoteric salts. They can be nonionic molecules, polar molecules, or molecular complexes capable of hydrogen bonding. The bioactive agent can be included in the compositions in the form of, for example, an uncharged molecule, a molecular complex, a salt, an ether, an ester, an amide, polymer drug conjugate, or other form to provide the effective biological or physiological activity.

Examples of bioactive agents that incorporated into systems herein include, but are not limited to, peptides, proteins such as hormones, enzymes, antibodies and the like, nucleic acids such as aptamers, iRNA, DNA, RNA, antisense nucleic acid or the like, antisense nucleic acid analogs or the like, low-molecular weight compounds, or high-molecular-weight compounds. Bioactive agents contemplated for use in the disclosed implantable composites include anabolic agents, antacids, anti-asthmatic agents, anti-cholesterolemic and anti-lipid agents, anti-coagulants, anti-convulsants, anti-diarrheals, anti-emetics, anti-infective agents including antibacterial and antimicrobial agents, anti-inflammatory agents, anti-manic agents, antimetabolite agents, anti-nauseants, anti-neoplastic agents, anti-obesity agents, anti-pyretic and analgesic agents, anti-spasmodic agents, anti-thrombotic agents, anti-tussive agents, anti-uricemic agents, anti-anginal agents, antihistamines, appetite suppressants, biologicals, cerebral dilators, coronary dilators, bronchiodilators, cytotoxic agents, decongestants, diuretics, diagnostic agents, erythropoietic agents, expectorants, gastrointestinal sedatives, hyperglycemic agents, hypnotics, hypoglycemic agents, immunomodulating agents, ion exchange resins, laxatives, mineral supplements, mucolytic agents, neuromuscular drugs, peripheral vasodilators, psychotropics, sedatives, stimulants, thyroid and anti-thyroid agents, tissue growth agents, uterine relaxants, vitamins, or antigenic materials.

Other bioactive agents include androgen inhibitors, polysaccharides, growth factors (e.g., a vascular endothelial growth factor-VEGF), hormones, anti-angiogenesis factors, dextromethorphan, dextromethorphan hydrobromide, noscapine, carbetapentane citrate, chlophedianol hydrochloride, chlorpheniramine maleate, phenindamine tartrate, pyrilamine maleate, doxylamine succinate, phenyltoloxamine citrate, phenylephrine hydrochloride, phenylpropanolamine hydrochloride, pseudoephedrine hydrochloride, ephedrine, codeine phosphate, codeine sulfate morphine, mineral supplements, cholestryramine, N-acetylprocainamide, acetaminophen, aspirin, ibuprofen, phenylpropanolamine hydrochloride, caffeine, guaifenesin, aluminum hydroxide, magnesium hydroxide, peptides, polypeptides, proteins, amino acids, hormones, interferons, cytokines, and vaccines.

Representative drugs that can be used as bioactive agents in the controlled release systems include, but are not limited to, peptide drugs, protein drugs, desensitizing materials, antigens, anti-infective agents such as antibiotics, antimicrobial agents, antiviral, antibacterial, antiparasitic, antifungal substances and combination thereof, antiallergenics, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, cardioactive agents, nonsteroidal anti-inflammatory agents, antiparkinsonian agents, antihypertensive agents, β-adrenergic blocking agents, nutritional agents, and the benzophenanthridine alkaloids. The agent can further be a substance capable of acting as a stimulant, sedative, hypnotic, analgesic, anticonvulsant, and the like.

The controlled release system can comprise a large number of bioactive agents either singly or in combination. Other bioactive agents include but are not limited to analgesics such as acetaminophen, acetylsalicylic acid, and the like; anesthetics such as lidocaine, xylocalne, and the like; anorexics such as dexadrine, phendimetrazine tartrate, and the like; antiarthritics such as methylprednisolone, ibuprofen, and the like; antiasthmatics such as terbutaline sulfate, theophylline, ephedrine, and the like; antibiotics such as sulfisoxazole, penicillin G, ampicillin, cephalosporins, amikacin, gentamicin, tetracyclines, chloramphenicol, erythromycin, clindamycin, isoniazid, rifampin, and the like; antifungals such as amphotericin B, nystatin, ketoconazole, and the like; antivirals such as acyclovir, amantadine, and the like; anticancer agents such as cyclophosphamide, methotrexate, etretinate, and the like; anticoagulants such as heparin, warfarin, and the like; anticonvulsants such as phenyloin sodium, diazepam, and the like; antidepressants such as isocarboxazid, amoxapine, and the like; antihistamines such as diphenhydramine HCl, chlorpheniramine maleate, and the like; hormones such as insulin, progestins, estrogens, corticoids, glucocorticoids, androgens, and the like; tranquilizers such as thorazine, diazepam, chlorpromazine HCl, reserpine, chlordiazepoxide HCl, and the like; antispasmodics such as belladonna alkaloids, dicyclomine hydrochloride, and the like; vitamins and minerals such as essential amino acids, calcium, iron, potassium, zinc, vitamin B₁₂, and the like; cardiovascular agents such as prazosin HCl, nitroglycerin, propranolol HCl, hydralazine HCl, pancrelipase, succinic acid dehydrogenase, and the like; peptides and proteins such as LHRH, somatostatin, calcitonin, growth hormone, glucagon-like peptides, growth releasing factor, angiotensin, FSH, EGF, bone morphogenic protein (BMP), erythopoeitin (EPO), interferon, interleukin, collagen, fibrinogen, insulin, Factor VIII, Factor IX, Enbrel®, Rituxam®, Herceptin®, alpha-glucosidase, Cerazyme/Ceredose®, vasopressin, ACTH, human serum albumin, gamma globulin, structural proteins, blood product proteins, complex proteins, enzymes, antibodies, monoclonal antibodies, and the like; prostaglandins; nucleic acids; carbohydrates; fats; narcotics such as morphine, codeine, and the like, psychotherapeutics; anti-malarials, L-dopa, diuretics such as furosemide, spironolactone, and the like; antiulcer drugs such as rantidine HCl, cimetidine HCl, and the like.

The bioactive agent can also be an immunomodulator, including, for example, cytokines, interleukins, interferon, colony stimulating factor, tumor necrosis factor, and the like; allergens such as cat dander, birch pollen, house dust mite, grass pollen, and the like; antigens of bacterial organisms such as Streptococcus pneumoniae, Haemophilus influenzae, Staphylococcus aureus, Streptococcus pyrogenes, Corynebacterium diphteriae, Listeria monocytogenes, Bacillus anthracis, Clostridium tetani, Clostridium botulinum, Clostridium perfringens. Neisseria meningitides, Neisseria gonorrhoeae, Streptococcus mutans. Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptspirosis interrogans, Borrelia burgddorferi, Campylobacter jejuni, and the like; antigens of such viruses as smallpox, influenza A and B, respiratory synctial, parainfluenza, measles, HIV, SARS, varicella-zoster, herpes simplex 1 and 2, cytomeglavirus, Epstein-Barr, rotavirus, rhinovirus, adenovirus, papillomavirus, poliovirus, mumps, rabies, rubella, coxsackieviruses, equine encephalitis, Japanese encephalitis, yellow fever, Rift Valley fever, lymphocytic choriomeningitis, hepatitis B, and the like; antigens of such fungal, protozoan, and parasitic organisms such as Cryptococcuc neoformans, Histoplasma capsulatum, Candida albicans, Candida tropicalis, Nocardia asteroids, Rickettsia ricketsii, Rickettsia typhi, Mycoplasma pneumoniae, Chlamyda psittaci, Chlamydia trachomatis, Plasmodium falciparum, Trypanasoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

In a further specific aspect, the bioactive agent comprises an antibiotic. The antibiotic can be, for example, one or more of Amikacin, Gentamicin, Kanamycin, Neomycin, Netilmicin, Streptomycin, Tobramycin, Paromomycin, Ansamycins, Geldanamycin, Herbimycin, Carbacephem, Loracarbef, Carbapenems, Ertapenem, Doripenem, Imipenem/Cilastatin, Meropenem, Cephalosporins (First generation), Cefadroxil, Cefazolin, Cefalotin or Cefalothin, Cefalexin, Cephalosporins (Second generation), Cefaclor, Cefamandole, Cefoxitin, Cefprozil, Cefuroxime, Cephalosporins (Third generation), Cefixime, Cefdinir, Cefditoren, Cefoperazone, Cefotaxime, Cefpodoxime, Ceftazidime, Ceftibuten, Ceftizoxime, Ceftriaxone, Cephalosporins (Fourth generation), Cefepime, Cephalosporins (Fifth generation), Ceftobiprole, Glycopeptides, Teicoplanin, Vancomycin, Macrolides, Azithromycin, Clarithromycin, Dirithromycin, Erythromycin, Roxithromycin, Troleandomycin, Telithromycin, Spectinomycin, Monobactams, Aztreonam, Penicillins, Amoxicillin, Ampicillin, Azlocillin, Carbenicillin, Cloxacillin, Dicloxacillin, Flucloxacillin, Mezlocillin, Meticillin, Nafcillin, Oxacillin, Penicillin, Piperacillin, Ticarcillin, Polypeptides, Bacitracin, Colistin, Polymyxin B, Quinolones, Ciprofloxacin, Enoxacin, Gatifloxacin, Levofloxacin, Lomefloxacin, Moxifloxacin, Norfloxacin, Ofloxacin, Trovafloxacin, Sulfonamides, Mafenide, Prontosil (archaic), Sulfacetamide, Sulfamethizole, Sulfanilimide (archaic), Sulfasalazine, Sulfisoxazole, Trimethoprim, Trimethoprim-Sulfamethoxazole (Co-trimoxazole) (TMP-SMX), Tetracyclines, including Demeclocycline, Doxycycline, Minocycline, Oxytetracycline, Tetracycline, and others; Arsphenamine, Chloramphenicol, Clindamycin, Lincomycin, Ethambutol, Fosfomycin, Fusidic acid, Furazolidone, Isoniazid, Linezolid, Metronidazole, Mupirocin, Nitrofurantoin, Platensimycin, Pyrazinamide, Quinupristin/Dalfopristin, Rifampicin (Rifampin in U.S.), Tinidazole, or a combination thereof. In one aspect, the bioactive agent can be a combination of Rifampicin (Rifampin in U.S.) and Minocycline.

In certain aspects, the bioactive agent can be present as a component in a pharmaceutical composition. Pharmaceutical compositions can be conveniently prepared in a desired dosage form, including, for example, a unit dosage form or controlled release dosage form, and prepared by any of the methods well known in the art of pharmacy. In general, pharmaceutical compositions are prepared by uniformly and intimately bringing the bioactive agent into association with a liquid carrier or a finely divided solid carrier, or both. The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. Other pharmaceutically acceptable carriers or components that can be mixed with the bioactive agent can include, for example, a fatty acid, a sugar, a salt, a water-soluble polymer such as polyethylene glycol, a protein, polysachamide, or carboxmethyl cellulose, a surfactant, a plasticizer, a high- or low-molecular-weight porosigen such as polymer or a salt or sugar, or a hydrophobic low-molecular-weight compound such as cholesterol or a wax.

The controlled release system can be administered to any desired subject. The subject can be a vertebrate, such as a mammal, a fish, a bird, a reptile, or an amphibian. The subject of the herein disclosed methods can be, for example, a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered.

Also disclosed are medical devices comprising the polymer blends or the particles or controlled release systems made therefrom. In general, the medical device can be any medical device. For some medical devices, the disclosed blends can be useful to provide the device with a desired tackiness or adhesive property, including for use in non-bioactive agent containing devices and applications. In one aspect, the medical device is an implant device. The implant device can comprise any shape, such as a rod, a fiber, a cylinder, a bead, a ribbon, a disc, a wafer, a free-formed shaped solid, or a variety of other shaped solids. The implant devices can include, for example, implants for drug delivery, including drug delivery pumps; orthopedic implants, including spinal implants, implants for osseointegration or bone repair; medical stents, including stents with inherent drug delivery capability; prosthetic implants, including breast implants, muscle implants, and the like; dental implants; ear implants, including cochlear implants and hearing devices; cardiac implants including pacemakers, catheters, etc.; space filling implants; bioelectric implants; neural implants; internal organ implants, including dialysis grafts; defribrillators; monitoring devices; recording devices; stimulators, including deep brain stimulators, nerve stimulators, bladder stimulators, and diaphragm stimulators; implantable identification devices and information chips; artificial organs; drug administering devices; implantable sensors/biosensors; screws; tubes; rods; plates; or artificial joints. In a specific aspect, the medical device is a drug delivery device comprising the polymer blends or the controlled release systems together with a bioactive agent which can be released from the drug delivery device. For the above described medical devices, useful polymer blends include without limitation those comprising lactide, glycolide, caprolactone, or a combination thereof (e.g. a copolymer thereof), among others.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in degrees Centigrade (° C.) or is at ambient temperature, and pressure is at or near atmospheric. There are numerous variations and combinations of reaction conditions, e.g., component concentrations, component mixtures, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

Microparticle formulations containing naltrexone base were prepared using an emulsion-based, solvent-extraction microencapsulation process as described below. Formulations were prepared using dissolved naltrexone base in the dispersed phase (DP) solutions. All biodegradable polymers were Lakeshore Biomaterials brand (SurModics Pharmaceuticals, Birmingham, Ala.).

A first batch was prepared (batch 1a) consisting of a single polymer, a poly(DL-lactide). A dispersed phase (DP) solution was prepared by dissolving 1.25 grams naltrexone base into 53.5 grams of polymer solution consisting of 7 wt % poly(DL-lactide) (0.37 dL/g) in ethyl acetate. The resulting DP solution was emulsified into 550 grams of a continuous phase (CP) solution consisting of 2 wt % aqueous polyvinyl alcohol (PVA) and containing 7.4 wt % ethyl acetate. Emulsification of the DP and CP was performed in a continuous manner by introducing the DP and CP solutions to the inlet port of a Silverson L4R-T mixer with inline attachment (speed setting 700 rpm). The flow rates for DP and CP solutions were 25 g/min and 250 g/min respectively. Microparticles were prepared by adding the emulsion directly to sufficient extraction phase (EP) water at an emulsion:EP water ratio of 1:7. The resulting suspension was collected into a container and stirred for 1 hour after which time the microparticle product was isolated by screening through 125 and 20 micron test sieves. The microparticles collected on the 20 micron sieve were washed with 2 L of de-ionized water.

After washing the microparticles were allowed to dry on the 20 micron sieve in a laminar flow hood.

A second batch (1b) which consisted of a blend of two different biodegradable polymers, δ 75:25 ratio (by weight) of a poly(DL-lactide) (as used in batch 1a) and a 75:25 poly(DL-lactide-co-glycolide). Batch 1b was made using a DP solution that was prepared by dissolving 1.25 grams naltrexone base into 53.5 grams of polymer solution consisting of 7 wt % total polymer concentration. For batch 1b, the polymer solution was prepared from a 75:25 blend (by weight) of a poly(DL-lactide) (0.37 dL/g) and a 75:25 poly(DL-lactide-co-glycolide) (0.42 dL/g) in ethyl acetate. Otherwise, this DP solution was used to prepare microparticles by the method described for batch 1a.

A third batch (batch 1c) was prepared from a polymer blend in a manner similar to batch 1b except that a 50:50 poly(DL-lactide-co-glycolide) 0.20 dL/g) was used in place of the 75:25 poly(DL-lactide-co-glycolide) to prepare the blended-polymer DP solution.

A fourth batch (batch 1d) was prepared from a polymer blend in a manner similar to batch 1b except that a PEG-block copolymer was used in place of the 75:25 poly(DL-lactide-co-glycolide) to prepare the blended-polymer DP solution. In this case, the PEG-block copolymer was prepared using a 1,500 dalton PEG (PEG-1,500) and the lactide-glycolide block was synthesized using a 65:35 ratio of lactide:glycolide (the PEG-block copolymer was a 65:35 poly(DL-lactide-co-glycolide-co-PEG-1,500) (0.46 dL/g)).

The drug content of the microparticle batches was determined by HPLC. A known amount of the microparticle formulation was dissolved into glacial acetic acid then phosphate-buffered saline (PBS) was added to precipitate the polymer. The sample was then filtered to remove polymer and the resulting solution was analyzed for naltrexone by HPLC using a Waters Nova-pak 3.9×150 mm column (Waters Corporation). Chromatographic conditions were as follows: 50 μL injection volume, UV detection at 280 nm, isocratic pump method involving sodium acetate buffer: methanol: triethylamine, 75:25:0.1 v/v/v.

In vitro release rates were characterized in triplicate by measuring naltrexone release into PBS at 37° C. using HPLC. A 20-30 mg sample was accurately weighed into a 50-mL glass test tube with conical bottom. Then 40-mL of PBS was then to the sample. The samples were incubated at 37° C. under shaking conditions (100 shakes per minute). At the specified time intervals, the samples were removed, mixed, and allowed to stand so the microparticles could settle to the bottom of the tube. Then a 5-mL sample was removed and was replaced by 5-mL of fresh PBS solution. The tubes were then placed back into the incubator until the next time point. The samples were analyzed by HPLC for drug content using the same method as described above. Cumulative percent naltrexone released was calculated as a mean and standard deviation.

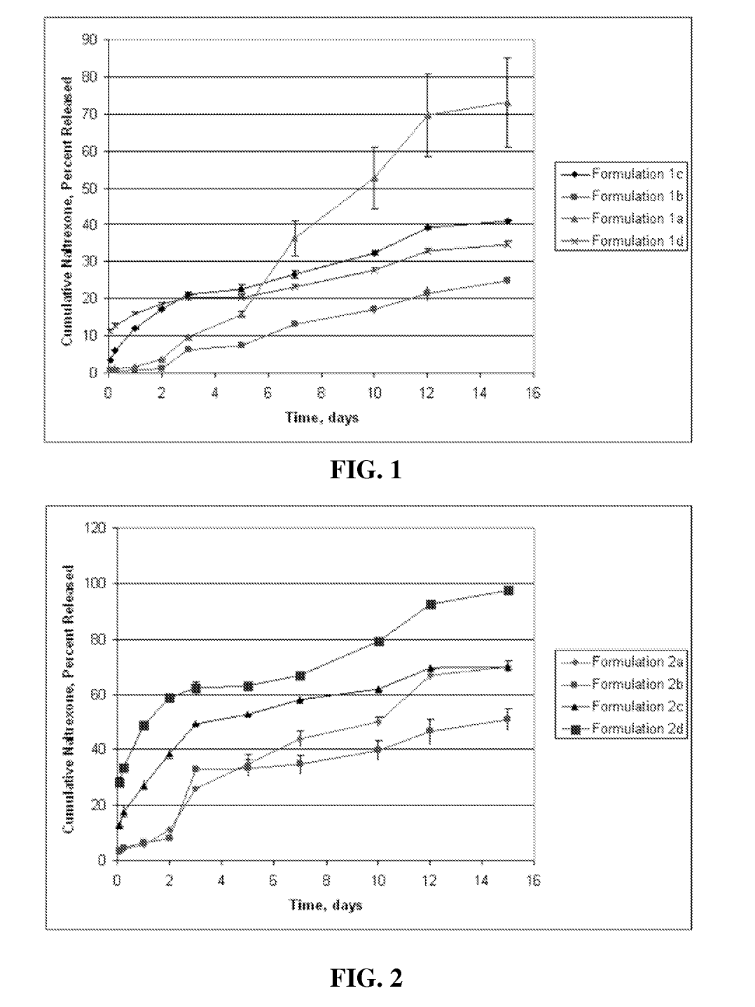

Drug loading and batch conditions are summarized in Table 1. The plot of drug release over time is shown in FIG. 1.

TABLE 1

Naltrexone loading, wt %

| Batch | TCL % | Actual |
|---|---|---|
| Batch 1a | 25 | 17.9 |
| Batch 1b | 25 | 19.6 |
| Batch 1c | 25 | 19.2 |
| Batch 1d | 25 | 8.2 |

### Example 2

Microparticle formulations containing naltrexone base were prepared using an emulsion-based, solvent-extraction microencapsulation process as described below. In these cases, formulations were prepared using excess dispersed naltrexone base in the dispersed phase (DP) solutions.

A dispersed phase (DP) solution was prepared by dissolving 0.3 grams naltrexone base into 19 grams polymer solution consisting of 20 wt % poly(DL-lactide) (0.37 dL/g) in ethyl acetate. An additional quantity of 0.95 grams of naltrexone base whose particle size had been ground to approximately 2 microns was then dispersed into this solution and was mixed with an IKA Ultra-Turrax T-25 mixer (with probe mixer attachment) (speed 3000 rpm) for 30 seconds. After mixing, the suspension was then stirred using a magnetic stir bar and stirring with a laboratory stir plate. The resulting DP solution (suspension) was emulsified into 250 grams CP solution consisting of 2 wt % aqueous PVA containing 7.4 wt % ethyl acetate. Emulsification of the DP and CP was performed in a continuous manner by introducing the DP and CP solutions to the inlet port of a Silverson L4R-T mixer with inline attachment (speed setting 1000 rpm). The flow-rates for DP and CP solutions were 25 g/min and 250 g/min respectively. Microparticles were prepared by adding the emulsion directly to sufficient extraction phase (EP) water at an emulsion:EP water ratio of 1:7. The resulting suspension was processed as described in Example 1, batch 1a. The resulting microparticle batch was labeled as batch 2a.

A second batch 2b was prepared using a blend of two biodegradable polymers. A dispersed phase (DP) solution was prepared by dissolving 0.3 grams naltrexone base into 19 grams polymer solution consisting of 20 wt % total polymer concentration in ethyl acetate. The polymer solution was prepared from a 75:25 blend (by weight) of a poly(DL-lactide) (0.37 dL/g) and 75:25 poly (DL-lactide-co-glycolide) (0.42 dL/g) polymer. An additional quantity of 0.95 grams of naltrexone base whose particle size had been ground to approximately 2 microns was then dispersed into this solution and was mixed as described previously. The resulting DP solution (suspension) was used to prepare microparticles as described for batch 2a.

A third batch, batch 2c, was prepared in a manner similar to batch 2b except that a 50:50 poly(DL-lactide-co-glycolide) (0.20 dL/g) was used in place of the 75:25 poly(DL-lactide-co-glycolide) polymer.

A fourth batch, batch 2d, was prepared in a manner similar to batch 2b except that a PEG-block copolymer, a 65:35 poly(DL-lactide-co-glycolide-co-PEG-1,500) (0.46 dL/g) was used in place of the 75:25 poly(DL-lactide-co-glycolide) polymer.

All samples were analyzed for drug content and in vitro release by methods described in Example 1.

Drug loading and batch conditions are summarized in Table 2. The plot of drug release over time is shown in FIG. 2.

TABLE 2

Naltrexone loading, wt %

| Lot no | TCL % | Actual |
|---|---|---|
| Batch 2a | 25 | 18.8 |
| Batch 2b | 25 | 23 |
| Batch 2c | 25 | 23.6 |
| Batch 2d | 25 | 9.9 |

Various modifications and variations can be made to the compounds, composites, kits, articles, devices, compositions, and methods described herein. Other aspects of the compounds, composites, kits, articles, devices, compositions, and methods described herein will be apparent from consideration of the specification and practice of the compounds, composites, kits, articles, devices, compositions, and methods disclosed herein. It is intended that the specification and examples be considered as exemplary.

What is claimed is:

1. A controlled release system comprising a polymer matrix comprising a first polymer and a second polymer that is different from the first polymer; and a bioactive agent encapsulated in the polymer matrix.

2. The controlled release system of claim 1, wherein the first polymer and the second polymer have different degradation rates in an aqueous medium.

3. The controlled release system of claim 1, wherein the first polymer and the second polymer have one or more different non-repeating units.

4. The controlled release system of claim 1, wherein the first polymer and the second polymer have one or more different end groups.

5. The controlled release system of claim 1, wherein the first polymer has a more polar end group than one or more end group(s) of the second polymer.

6. The controlled release system of claim 1, wherein the first polymer has a more polar end group than all end group(s) of the second polymer.

7. The controlled release system of claim 1, wherein the first polymer has one or more carboxylic acid end groups, and wherein the second polymer has one or more ester end groups.

8. The controlled release system of claim 1, wherein the first polymer and the second polymer have different molecular weights.

9. The controlled release system of claim 1, wherein the first polymer has a molecular weight that is at least about 3000 Daltons greater than the molecular weight of the second polymer.

10. The controlled release system of claim 1, wherein the first polymer exhibits a glass-transition temperature that is less than the glass-transition temperature exhibited by the second polymer.

11. The controlled release system of claim 1, wherein the first polymer exhibits a glass-transition temperature that is from about 5° C. to about 50° C. less than the glass-transition temperature exhibited by the second polymer.

12. The controlled release system of claim 1, wherein the controlled release system further comprises a third polymer that is different from the first and second polymers.

13. The controlled release system of claim 1, wherein the first and second polymers are both poly(lactide-co-glycolide) polymers.

14. The controlled release system of claim 1, wherein the first and second polymers are both poly(lactide-co-glycolide) polymers; wherein the ratio of lactide to glycolide is from about 90:10 to about 40:60.

15. The controlled release system of claim 1, wherein the first and second polymers are both poly(lactide-co-glycolide) polymers; wherein the ratio of lactide to glycolide is from about 85:15 to about 50:50.

16. The controlled release system of claim 1, wherein the controlled release system is an implant device or a microparticle.

17. The controlled release system of claim 1, wherein the controlled release system is a bioactive agent-loaded rod.

18. The controlled release system of claim 1, wherein the first polymer is poly(lactide), and the second polymer is poly(lactide-co-glycolide) having a ratio of lactide to glycolide of about 75:25; wherein the ratio of the first polymer to the second polymer is about 75:25.

19. The controlled release system of claim 1, wherein the first polymer is poly(lactide), and the second polymer is poly(lactide-co-glycolide) having a ratio of lactide to glycolide of about 50:50; wherein the ratio of the first polymer to the second polymer is about 75:25.

20. The controlled release system of claim 1, wherein the first polymer is poly(lactide), and the second polymer is polyethylene glycol (PEG) having a molecular weight of about 1500 Daltons; wherein the ratio of the first polymer to the second polymer is about 75:25.
